(19) United States
(12) Patent Application Publication
(10) Pub. No.: US 2013/0226269 A1
(43) Pub. Date: Aug. 29, 2013

(54) DISPOSABLE PATCH FOR PERSONAL AESTHETIC SKIN TREATMENT

(71) Applicants: Shimon Eckhouse, Haifa (IL); Lion Flyash, Nazareth Illit (IL)

(72) Inventors: Shimon Eckhouse, Haifa (IL); Lion Flyash, Nazareth Illit (IL)

(73) Assignee: SYNERON MEDICAL LTD, Yoqneam Illit (IL)

(21) Appl. No.: 13/853,057

(22) Filed: Mar. 29, 2013

Related U.S. Application Data
(63) Continuation of application No. PCT/IL2011/000781, filed on Oct. 6, 2011.
(60) Provisional application No. 61/427,177, filed on Dec. 25, 2010.
(60) Provisional application No. 61/427,305, filed on Dec. 27, 2010.
(60) Provisional application No. 61/393,902, filed on Oct. 17, 2010.

(51) Int. Cl. A61N 1/04; A61N 5/06

(52) U.S. Cl. CPC A61N 1/0496; A61N 5/0616
USPC 607/88; 607/148

(57) ABSTRACT

An apparatus for personal aesthetic skin treatment by RF voltage. The apparatus includes an RF voltage supply and a disposable patch with an assembly of individual electrodes operative to contact segments of the skin and deliver to each contact RF voltage. The RF voltage may be supplied to each of the electrodes according to a predetermined experimentally established skin treatment protocol. The treatment RF current generated by the applied RF voltage heats the skin and is applied intermittently to different electrodes being in contact with the skin in an order and duration sufficient to cause the desired skin effect and enable proper cooling of earlier treated skin segments. The selected protocol ensures safe non-ablative skin treatment parameters.

### CROSS-REFERENCE TO RELATED APPLICATIONS

This application is being filed under 37 U.S.C. 111 as a continuation application of International Application Number PCT/IL2011/000781, which has an international filing date of Oct. 6, 2011 and which claims priority to the following U.S. provisional applications for patent: Ser. No. 61/393,902 filed on Oct. 17, 2010, Ser. No. 61/427,305 filed on Dec. 27, 2010 and Ser. No. 61,427,177 filed on Dec. 27, 2010. This application claims the benefit of the priority date Oct. 17, 2010 under 37 U.S.C. 120 as a continuation of PCT/IL2011/000781 which claims priority as previously stated. The International Application Number PCT/IL2011/000781 is co-pending at the filing of this application and includes at least one common inventor. This application incorporates the above-identified International Applications and United States Provisional applications by reference in their entirety.

### TECHNOLOGY FIELD

The present apparatus is related to the field of personal aesthetic procedures and in particular to cosmetic skin treatment procedures.

### BACKGROUND

Skin tightening or wrinkle reduction, removal of skin lesions and blemishes, reduction of subcutaneous fat or adipose tissue, are aesthetic treatments for which there is a growing demand. Types of available aesthetic therapy commonly include the application of different light sources, radio frequency energy and sometimes ultrasound energy.

The electromagnetic energy is typically delivered to a target segment of the skin of a recipient by selecting a contact element that is compatible with the treated skin segment size. Alternatively, a plurality of contact elements may be utilized, in which the plurality of elements contact discrete points of the target segment of the skin. In the latter case, the healing period is typically shorter. Although both modes of treatment are effective, the use of multiple contact elements treating discrete points or fractions of a target skin segment effectively tightens the skin, reduces wrinkles, and improves the skin appearance. In recent years, non-invasive, non-ablative aesthetic skin treatments have been introduced and may replace ablative skin treatment procedures in the future. In non-ablative skin treatment, thermal energy induces certain tissue modification and in particular collagen modification in the dermis. Currently non-ablative skin treatment is used for skin tightening, scar removal, acne treatment, and other aesthetic procedures typically performed in an ambulatory environment.

In non-ablative skin treatment radiofrequency (RF) energy, depending on the spacing of electrodes, is deposited 100-2500 μm below the skin surface, where the energy does not affect the epidermis and the skin layer in which most of the skin aging processes occur. With no epidermal wound, there is almost no recovery period and thus no interruption of daily life routines. Transient erythema or mild edema, are the only known side effects and those disappear a few hours after the treatment. The efficiency of the non-ablative treatments is lower than the one of ablative treatments; however, non-ablative skin treatments also stimulate new collagen production and repair tissue defects.

Since there are no side effects and the procedure does not leave wounds requiring a long healing period, the non-ablative treatment is associated with little or no downtime and unlike the ablative skin treatment, which requires professional supervision, non-ablative skin treatment may be used by a lay user in a home environment at a time most convenient for him/her to perform a treatment session such as, for example, skin tightening and wrinkle reduction associated with collagen remodeling.

RF energy is conducted to skin through electrodes. With proper design of RF applying electrodes, RF energy power setting and application time the energy may be accurately conducted to the desired target tissue. For example, the energy application time and power may be shorter than skin thermal relaxation time further simplifying the non-ablative skin treatment. The employment of an applicator that includes disposable parts for electromagnetic radiation skin treatment also simplifies and facilitates aesthetic treatments in a home environment at a time most convenient for the user to perform a treatment session.

Use of RF energy for performing skin treatment be a lay user in a residential environment as compared to professional use devices requires increased safety, reduced device size and freedom to perform concurrently to the treatment other tasks.

### BRIEF SUMMARY

The apparatus for personal aesthetic skin treatment by RF voltage includes an RF voltage supply and a disposable patch with an assembly of individual electrodes operative to contact segments of the skin and deliver to each skin segment being in contact with the electrodes RF voltage. The RF voltage may be supplied individually to each of the electrodes, to a group of electrodes, and to all electrodes of the patch according to a predetermined experimentally established protocol. The treatment RF current generated by the applied RF voltage heats the skin and is applied intermittently to different electrodes being in contact with the skin in an order and duration sufficient to cause the desired skin effect and enable proper cooling of earlier treated skin segments. The selected protocol ensures safe non-ablative skin treatment parameters.

Typically, the electrodes are assembled on a common substrate or carrier that may be a reusable or disposable carrier. One or more light sources providing illumination to the treated skin segment may be assembled on the same substrate. The light sources may be operative to illuminate the treated skin segment independent of the RF voltage application, concurrent with the RF voltage application or in sequentially with RF voltage application.

### Glossary

The term “patch” in the context of the present disclosure means a substrate having an array of voltage to skin application elements or electrodes. The electrodes may be in the form of one or more rows of voltage to skin application elements, a two dimensional array or matrix of voltage to skin application elements and a three-dimensional shape substrate having on the surface to be applied to the skin voltage to skin application elements. In addition to the electrodes the patch may include light source, for example surface mounted LEDs or fiber optics lines.

The terms “electrodes”, “conductive elements”, “contact elements” and “voltage to skin application elements” are used interchangeably in the present disclosure and mean elements operative to receive voltage from a source such as, for example, an RF voltage generator and apply the received voltage to the skin.

The term “skin treatment” as used in the present disclosure includes cosmetic treatment of various skin layers such as stratum corneum, dermis, epidermis, skin rejuvenation procedures, pigmented lesions removal, acne treatment, and such procedures as collagen shrinking or destruction. The terms “RF voltage” and “RF power” are used interchangeably in the present disclosure. The mathematical relation between these two parameters is well known and knowledge of the value of one of them enables easy determination of the value of the other parameter.

### BRIEF DESCRIPTION OF THE DRAWINGS

Various embodiments of the present apparatus, including method and apparatus embodiments, are disclosed and presented, by way of non-limiting examples only, with reference to the accompanying drawings, wherein like numerals depict the same elements throughout the text of the specifications. The present apparatus and skin treatment method will be understood and appreciated more fully from the following detailed description, taken in conjunction with the drawings in which:

FIG. 1 is a simplified illustration of an exemplary embodiment of the present apparatus for personal aesthetic skin treatment;

FIGS. 2A, 2B, 2C and 2D are plan view simplified illustrations of some exemplary embodiments of disposable RF to skin application patches;

FIG. 2E is a side view taken from view K in FIG. 2A.

FIG. 3 is a planar view simplified illustration of a sheet of electrodes containing a number of exemplary patch shapes in accordance with the present method and apparatus;

FIGS. 4A and 4B are simplified illustrations of an exemplary embodiment of cosmetic skin treatment in accordance with the present method and apparatus;

FIGS. 5A and 5B are frontal and rear planar views illustrating an additional exemplary embodiment of a disposable skin treatment patch in accordance with the present method and apparatus;

FIGS. 6A and 6B are plan view simplified illustrations of another exemplary embodiment of disposable RF to skin application patch;

FIGS. 7A, 7B, 7C and 7D are plan view simplified illustrations of generation of a linear sweeping heating wave effect according to an exemplary embodiment of the present method; and

FIG. 8 is a simplified illustration of a user performing skin treatment in accordance with the current method and apparatus.

### DETAILED DESCRIPTION OF EXEMPLARY EMBODIMENTS

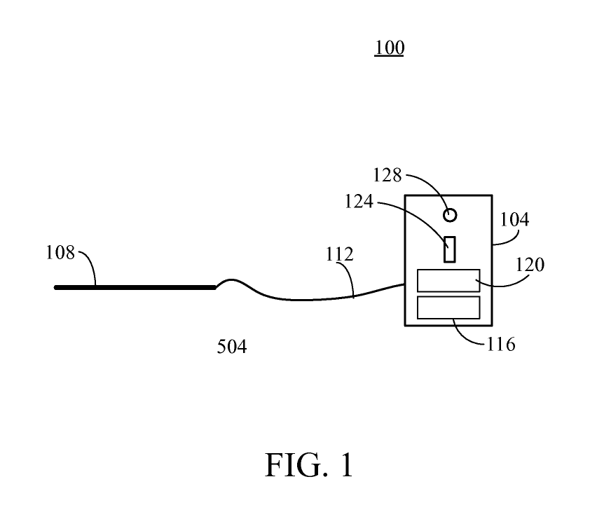

Reference is made to FIG. 1, which is a simplified illustration of an exemplary embodiment of the present apparatus for personal aesthetic skin treatment. As shown in FIG. 1, apparatus 100 includes a case 104, a disposable RF to skin application patch 108 and a cable 112 connecting between case 104 and patch 108. Case 104 may contain a source of power 116, an RF voltage generator 120, an ON-OFF switch or button 124, and an operation status indicator 128 such as a Light Emitting Diode (LED) that may lit with one or more colors. The ON-OFF switch may include more than one button enabling selection of a number of treatment protocols. The source of power 116 may be one or more of conventional batteries that are disposed upon depletion or one or more of rechargeable batteries. The distal end of cable 112 is equipped by a fast release connector (not shown) enabling easy connection between case 104 and disposable RF to skin application patch 108.

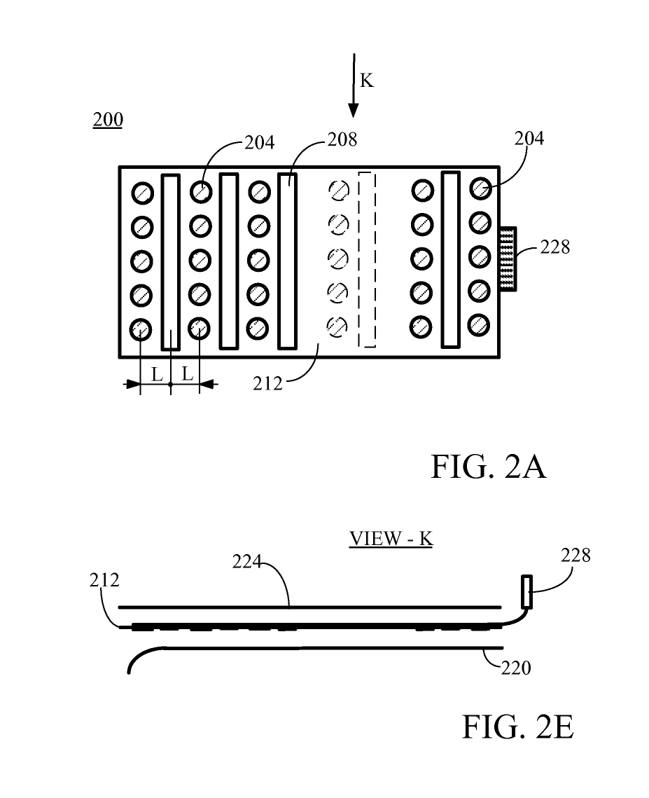

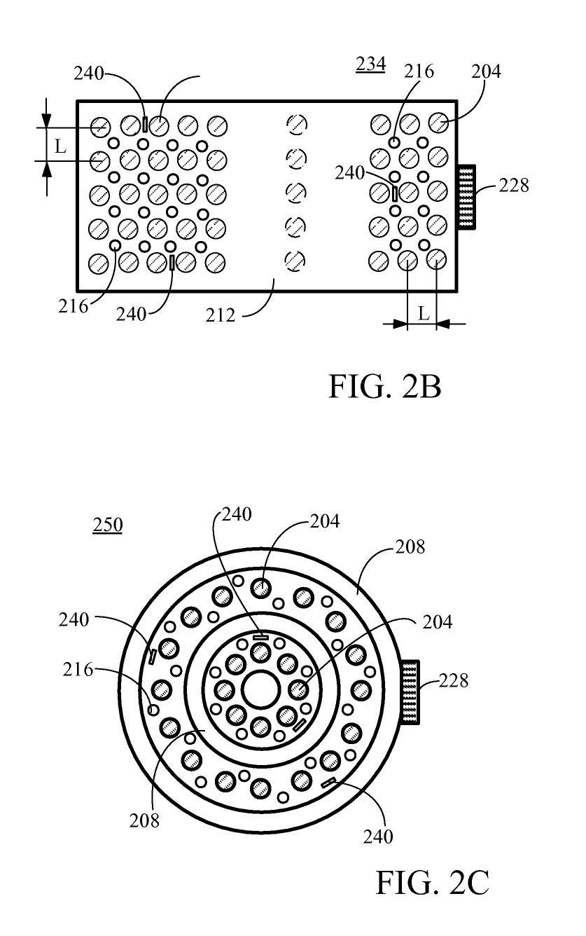

FIGS. 2A, 2B, 2C and 2D are plan view simplified illustrations of some exemplary embodiments of disposable RF to skin application patches. FIG. 2A illustrates a rectangular patch 200 that may be used instead of patch 108. Generally, patches 108, 200 and other patches to be described below are multilayer structures where one or more electrodes or voltage to skin application elements 204 and 208 are deposited on a substrate 212. The voltage to skin application elements or electrodes 204 and 208 may be coated by a coating enhancing certain electrode surface properties. For example, the coating may be an adhesive coating including an electrically conductive biocompatible adhesive, for example, such as Conductive Adhesives for Biomedical Electrodes commercially available from First Water Limited, Ramsbury, Wiltshire SN8 2RB U.K. or conductive, pressure sensitive adhesive commercially available from Avery Dennison, Inc., Painesville, Ohio 44077 U.S.A. Such adhesive would enable firm electrical and mechanical coupling of the electrode to the skin when the patch is applied to the skin.

FIG. 2E is a side view taken from view K in FIG. 2A. A release layer 220, which may be a suitable paper or plastic material, protecting the adhesive may be attached to the electrodes. The release layer may cover the whole surface of the patch. The other side, of substrate 212 on which electrodes interconnecting pattern is deposited, may be covered by a protective layer 224. Protective layer 224 may be a plastic or paper layer of a desired color or a lacquer layer. The color of the protective layer may be used as a code indicating patch 200 skin treatment parameters.

In another embodiment protective layer 224 may be a thermally conductive material, since some of the heat developed at the skin-electrode contact surface will be conducted through the electrodes to the conductors interconnecting pattern deposited on the opposite to the electrodes side of the substrate. Such material, with proper electrical isolation means, may be aluminum or copper foil, metal powder included in the material of layer 224 or similar materials. The heat dissipation capability of the foil may be enhanced by providing a structure of fine ribs or fine rough structure of the heat dissipating surface of coating layer 224. At least one edge of substrate 212 may have an extension 228 or a bay enabling easy and quick connection to RF voltage generator 124 located in case 104 (FIG. 1) by cable 112 (FIG. 1).

Patch 200 of FIG. 2A has electrodes layout where one or more electrodes 204 located on both sides of a common electrode 208. This electrodes layout enables patch operation is similar to mono-polar RF operation mode. The spacing between electrodes 204 and 208 is uniform and electrodes 204 located on both sides of electrode 208 may be located on the same distance from electrode 208. Optionally, the spacing between the electrodes may be coated by an electrically insulating adhesive, which ensures better patch to skin adhesion and stability. Uniform spacing L between the electrodes enables treatment of all skin segments, actually skin volumes located below the skin segments surface, to which the patch is applied at the same skin treatment depth. For treatment of skin layers located at other skin depth patches with different spacing or distance between the electrodes may be used. Alternatively, a different electrode switching pattern enabling treatment of skin layers located at different depth may be implemented.

FIG. 2B illustrates a patch 234 configured to operate in bi-polar operation mode. Common electrodes 208 have been replaced by electrodes 204 located on a grid having equal step/spacing L in both grid directions, although other asymmetric electrodes spacing is possible. Optionally, surface mounted LEDs 216 may be incorporated in the patch. Alternatively, an LED or other suitable light source may be included into case 104 and a fiber optic bundle, which may be incorporated into cable 112, may guide the light to the treated skin segment. LEDs 216 or the light source may emit radiation with wavelength of 570-780 nm.

Voltage to skin applying elements or electrodes may be produced by different methods. Typically, methods used in printed circuit board production may be suitable for voltage to skin applying elements or electrodes production. These methods enable low cost production of a large amount of substrates populated by electrodes. Depending on the type of processing and material deposition the voltage to skin applying elements may be flat, protruding from the surface on few microns or more as desired. By proper selection of the metal deposition process the voltage to skin applying elements may be made flat or have a certain shape and surface texture. The substrate 212 on which the electrodes 204 and 208 and LEDs 216 reside is common to all electrodes and may be made of a variety of materials, typically insulating materials. Non-limiting example of a suitable substrate materials include polyimide film, paper, or similar material, with a thickness of 0.5 mil to 60 mil (12.5 micron to 1500 micron). All described above patches are configured to include an extension 228 or similar connector type arrangement allowing quick attachment to apparatus 100.

The patches may be of different geometrical shapes and sizes. The shape of the patches may resemble the skin segment to which the patches may be applied, e.g. under eyes, on the neck, etc. FIG. 2C illustrates a round shape patch 250. The patch may be configured to operate in bi-polar operation mode also. Common electrodes 208 could be replaced by electrodes 204 located on a grid having equal or different step in both grid directions. FIG. 2D illustrates a patch 256 having a shape suitable for example, for application under eyes.

In some embodiments, each of the patches may include one or more temperature sensors 240, which may be a thermistor, a thermocouple or a thin film sensor.

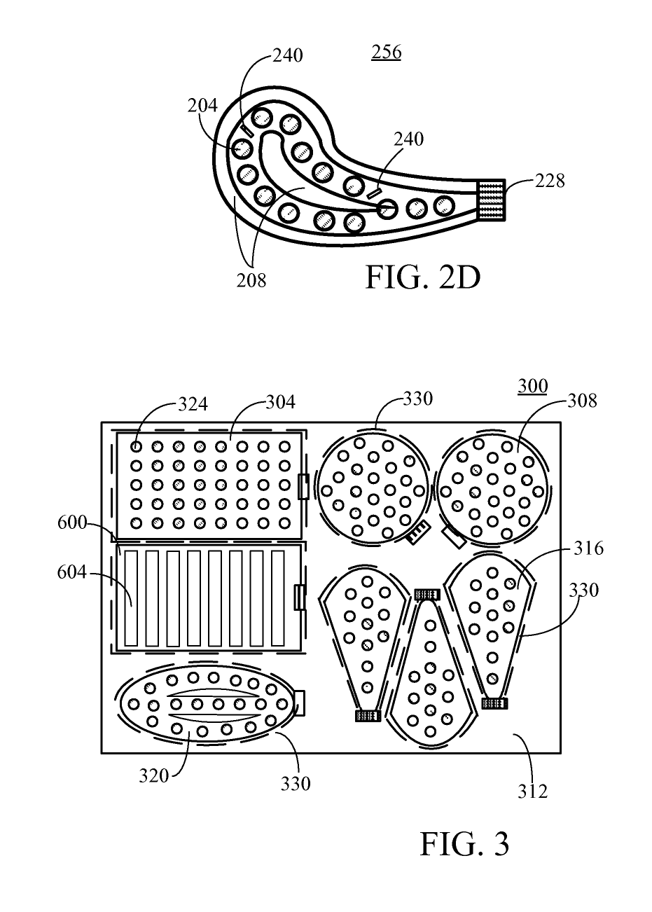

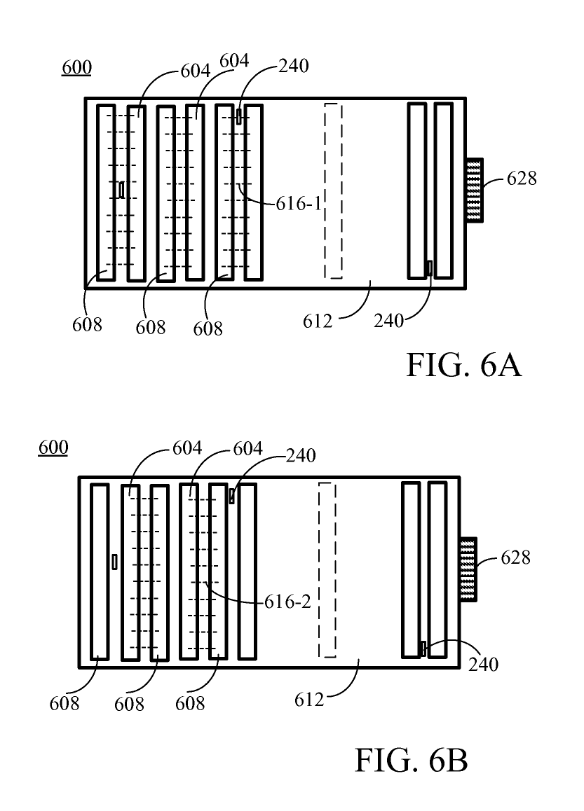

The patches for personal fractal cosmetic skin treatment may be supplied to the user in single units according to the desired predetermined patch shape. Alternatively, as shown in FIG. 3, the patches may be supplied in substrate sheets 300. The substrates 312 of sheets 300 are made of the same material as substrate 212 and each substrate may contain a plurality of different or identical patch shapes and sizes, for example, such shapes as shapes 304, 308, 316, and 320. Different or identical electrode 324 configurations may be placed on each of the patch shapes. (For the simplicity of the explanation all of the patches, except patch 600, are shown with similar electrodes.) A layer of electrically conductive adhesive (not shown) covers the surface of each of the electrodes located on the patch. The space between electrodes may be covered by an electrically isolating adhesive. A release layer may cover the sheet of disposable patches. Cut-out lines 330 enabling easy patch 304, 308, 316, 320 or 600 (FIG. 6) from sheet 300 separation could be made in the substrate 312 and in the release layer. Supply of skin treatment patches in sheets containing a plurality of shapes reduces distribution and manufacturing cost and provides the user with a greater patch selection freedom.

The size of the patches may vary and may be adapted to the size of treated skin segment. The patches may be small for example 100 mm×10 mm for localized skin treatment or large enough, for example 100 mm by 100 mm to treat relatively large skin segments.

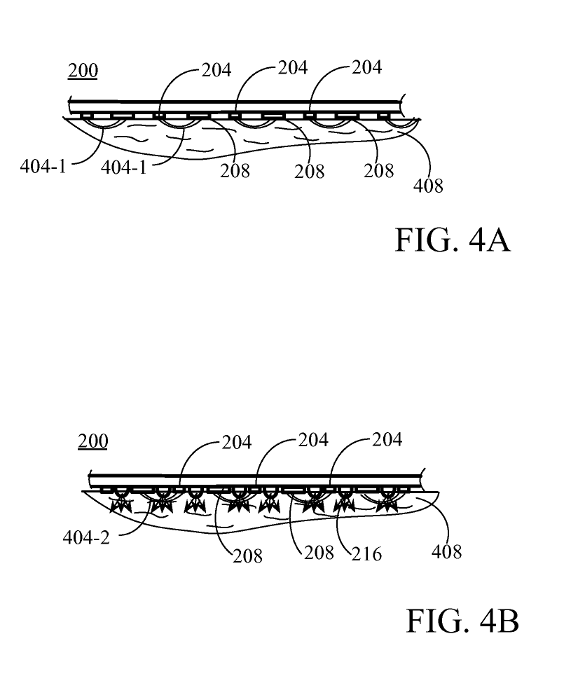

FIGS. 4A and 4B are simplified illustrations of an exemplary embodiment of cosmetic skin treatment in accordance with the present method and apparatus. Initially the user applies patch 200, or any other of the patches described above, to a segment of the user skin to be treated and checks that the electrodes are in firm contact with the skin. The user connects between patch 200 and case 104 by cable 112 and by pushing button 124 (FIG. 1) switches ON RF voltage generator 120 and may set one of the predetermined skin treatment protocols. RF voltage generator provides test RF voltage and determines the quality of the contact between the patch electrodes and the treated segment of skin. Upon determination of the electrodes status RF voltage generator may begin delivery of the treatment RF voltage to the electrodes of the patch. The user may select a proper treatment protocol by using the ON-OFF switch and keying in the protocol number.

The magnitude of test RF voltage supplied to the electrodes is set between 10 vrms to 30 vrms and of the treatment voltage between 20 vrms to 200 vrms such as not to cause any excessive and damaging skin heating. The thermal properties of the skin are sufficiently predictable; the effect of treatment can be estimated from previous measurements made in laboratory conditions. These measurements may be a basis for predetermined skin treatment protocols according to which the RF voltage generator will operate. The lengths of the intervals (and the time between the intervals) in course of which RF voltage generator 120 (FIG. 1) may deliver RF voltage and induce in skin 408 RF current could be controlled without having direct temperature feedback by “dosing” the intervals or pulse length of the RF voltage. For example, the pulses may have duration of 0.5sec to 4sec or even be set to operate in pseudo continuous mode. Proper dosing may be included in each of the predetermined skin treatment protocols. Alternatively, temperature sensors 240 (FIG. 2) may be operative to switch OFF the supply of RF voltage when the skin or electrode temperature exceeds the desired or preset limit.

In order to further mitigate potential skin overheating, initially RF voltage may be delivered to common electrodes 208 (FIGS. 2A, 2B, 2C and 2D) and to all fractal electrodes located on one side (for example, on the left or first side or in the inner circle (FIG. 2C) of the common electrode 208 creating in the skin 408 an electric current schematically shown by lines 404-1 (FIG. 4A).

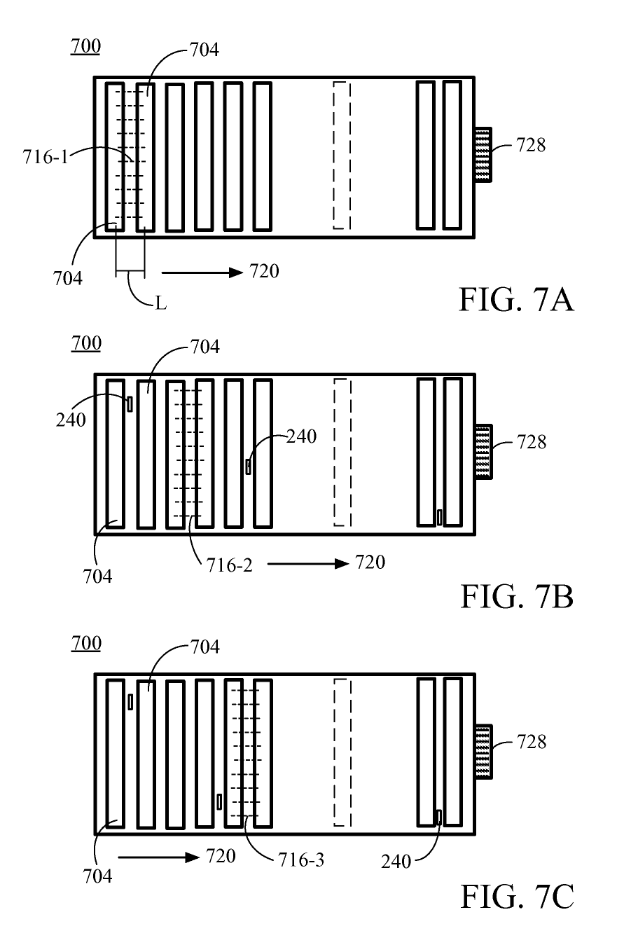

Upon completion of delivering of RF voltage to the first group of electrodes, RF voltage generator may switch-off the first group of electrodes and begin delivery of the RF voltage to the same common electrodes 208 and another group of fractal electrodes 204, for example, electrodes located on the right or second side or in the outer circle (FIG. 2C) of the common electrode 208 creating in the skin 408 an electric current schematically shown by lines 404-2 (FIG. 4B). This mode of treatment enables fractal treatment of densely placed skin treatment points and reduces the risk of skin overheating. The earlier treated fractions of skin are thermally relaxing or cooling when the next fraction of skin is treated. (Skin thermal relaxation time is known to vary from few milliseconds to few seconds depending on the depth of the treatment.) FIG. 4B illustrates optional LEDs 216 mounted on patch 200 and operative to illuminate the treated segment of skin 408. Alternatively, terminations of fiber optics guides emitting light from a source of illumination may be mounted on patch 200. LEDs 216 or fiber optics terminations may illuminate the treated segment of skin 408 concurrently with the application of RF voltage, before the application of the RF voltage or after the application of RF voltage. Coating layer 224 assists in reducing temperature of the skin surface. A similar mode or operation may be applied to patches with fractal only electrodes (FIG. 2B) or conventional electrodes 604 and 704 (FIG. 6 and FIG. 7).

Patch electrodes 204 and 208 (FIG. 2) are in permanent engagement or contact with skin while the RF current is supplied in pulses to increase the temperature of the treated skin segments or volumes under the skin surface to about 60-62 degrees Celsius and maintain it for a certain treatment time, although limiting heating of the skin surface to 40-45 degrees Celsius or a lower value. As indicated earlier the treatment mode and treatment parameters may be determined earlier in laboratory conditions and applied/configured by the patch user. Although, the treatment parameters may be determined earlier in laboratory conditions and applied by the patch user, temperature sensors 240 may be activated to control the skin and electrode temperature and if necessary switch-OFF RF voltage to electrodes supply.

In use case 104 may be placed in a pouch located on the user waist or hand, similar to the way iPod and other music playing apparatuses are carried. This enables complete user freedom that in course of treatment may address and work on other issues and tasks.

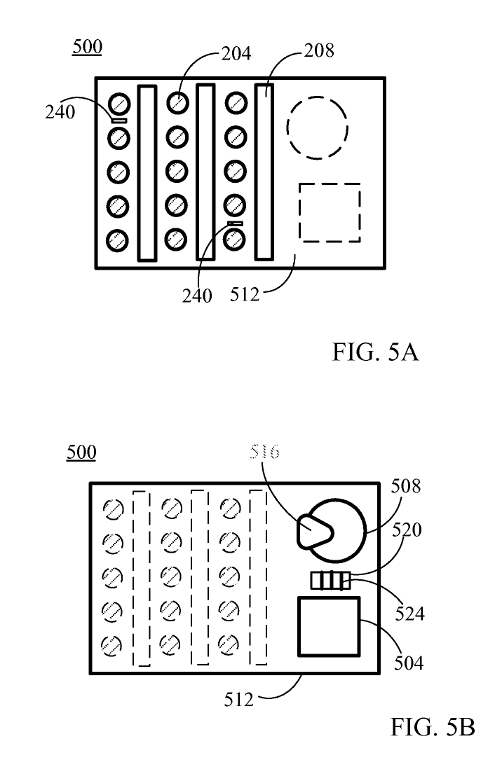

FIG. 5A and 5B are frontal and rear planar views illustrating an additional exemplary embodiment of a disposable skin treatment patch in accordance with the present method and apparatus. Depending on the amount of electrodes on the substrate 512, patch 500 that may be used instead of patch 104 or 200 or any other described above patches. Patch 500 and any other described in the present disclosure patch may be implemented as a disposable patch. The patch could include an RF voltage generator 504 and a power source such as a battery 508 and as such the patch becomes an autonomous patch that does not require connection to a power supply. The electrodes 204 and 208 of the patch 500 are coated by an electrically conductive adhesive enabling a firm electrical and mechanical coupling of the electrode to the skin. When patch 500 is applied to skin a current begins flowing in the skin. The current is sensed by a current sensor and if all of the electrodes are in firm contact with the skin, it switches ON RF voltage generator 504 and supplies treatment voltage to the skin.

RF voltage generator 504 of patch 500 may operate according to a single or a number of skin treatment protocols. When patch 500 is designed to operate in a number of skin treatment protocols it includes an optional skin treatment protocol setting device 520. In order to instruct the patch/RF voltage generator to operate the desired skin treatment protocol, the user configures the skin treatment protocol setting device 520 by simply cutting one or more conductors 524 leaving the one or a combination of conductors that enables the desired skin treatment protocol. As a safety measure temperature sensors 240 may be mounted on patch 500 substrate and used to switch-OFF RF voltage supply.

FIGS. 6A and 6B are plan view simplified illustrations of another exemplary embodiment of a disposable RF to skin application patch. FIG. 6A illustrates a rectangular patch 600 that may be used instead of patch 108. The patch is a multilayer structure where one or more conventional electrodes or voltage to skin application elements 604 and 608 are deposited on a substrate 612. (Electrodes 608 and 604 may be identical electrodes, although for convenience of explanation they have been given different numerals.) The voltage to skin application elements or electrodes 604 and 608 may be coated by an electrically conductive biocompatible adhesive. Such adhesive would enable firm electrical and mechanical coupling of the electrode to the skin when the patch is applied to the skin. The other side, of substrate 612 on which electrodes interconnecting pattern is deposited, may be covered by a protective layer, which may be a plastic or paper layer of a desired color or a lacquer layer.

In another embodiment protective layer may be a thermally conductive material, since some of the heat developed at the skin-electrode contact surface will be conducted through the electrodes to the electrodes interconnecting pattern deposited on the opposite to the electrodes side of the substrate. Such material may be aluminum or copper foil, metal powder included in the material of the protective layer. One or more thermal sensors 240 may also be located on the patch. At least one edge of substrate 612 may have an extension 628 or a bay enabling easy and quick connection to RF voltage generator 120 located in case 104 (FIG. 1) by cable 112 or similar cable.

Patch 600 has a layout of electrodes where one or more electrodes 608 located on both sides of electrode 604. The spacing between electrodes 604 and 608 is uniform and electrodes 608 located on both sides of electrode 604 may be located on the same distance from electrode 604. Uniform spacing between the electrodes enables treatment of all skin segments to which the patch is applied at the same skin treatment depth. For treatment of skin layers located at a different skin depth, patches with different spacing or distance between the electrodes may be used or the RF voltage switching order between the electrodes may be changed.

In order to reduce the risk of potential skin overheating, initially RF voltage may be delivered to electrodes 608 and 604 located on one side (for example, on the left or first side of electrode 604 creating in the skin an electric current schematically shown by lines 616-1. As a safety measure temperature sensors 240 may be mounted on patch 600 substrate and used to switch-OFF RF voltage supply.

Upon completion of delivering of RF voltage to the first group of electrodes 604 and 608, RF voltage generator may switch off the first group of electrodes and begin delivery of the RF voltage to another group of electrodes 604 and 608, for example, electrodes located on the right or second side of electrode 604 creating in the skin an electric current schematically shown by lines 616-2. This mode of treatment reduces the risk of skin overheating. The earlier treated segments of skin are thermally relaxing or cooling when the next segment of skin is treated. Coating layer, deposited on the back surface of the electrode assists in reducing temperature of the skin surface.

Referring now to FIGS. 7A-7D which are plan view illustrations of generation of a linear sweeping heating wave effect by conventional RF electrodes 704 similar to electrodes 604 across patch 700 in accordance with an exemplary embodiment of the current method and apparatus having extension 728. In FIG. 7A, only the first two electrodes 704 are activated generating a tissue heating effect in a skin segment marked 716-1 that could be located between the electrodes. In FIG. 7B the first two electrodes 704 are inactivated and the next two electrodes 704 are activated generating a tissue heating effect in a skin segment marked 716-2 and so on. Activating electrodes 704 as described in detail above, may create a linear sweeping tissue heating wave effect moving the treated skin segment in a direction indicated by arrows 720.

In FIG. 7C, all previous pairs of electrodes are inactivated and only electrodes 704 of the third pair of electrodes are activated generating a tissue heating effect in a skin segment marked 716-3. In FIG. 7D more than one pair of electrodes 704 is activated concurrently. The distance between active electrode pairs may be selected to enable thermal relaxation of the treated tissue.

The treatment protocols including the treatment mode and treatment parameters may be determined earlier in laboratory conditions. Patch electrodes disclosed above are in permanent engagement/contact with skin while the RF current is supplied in pulses pulsed to increase the temperature of the treated skin volume to about 40-62 degrees Celsius and maintain it for a certain treatment time, although limiting heating of the skin surface to 40-45 degrees Celsius or a lower value. As it is known in the art, methods of cooling both electrodes and skin surface exist and are described elsewhere. Any of these cooling methods may be applied with the present treatment.

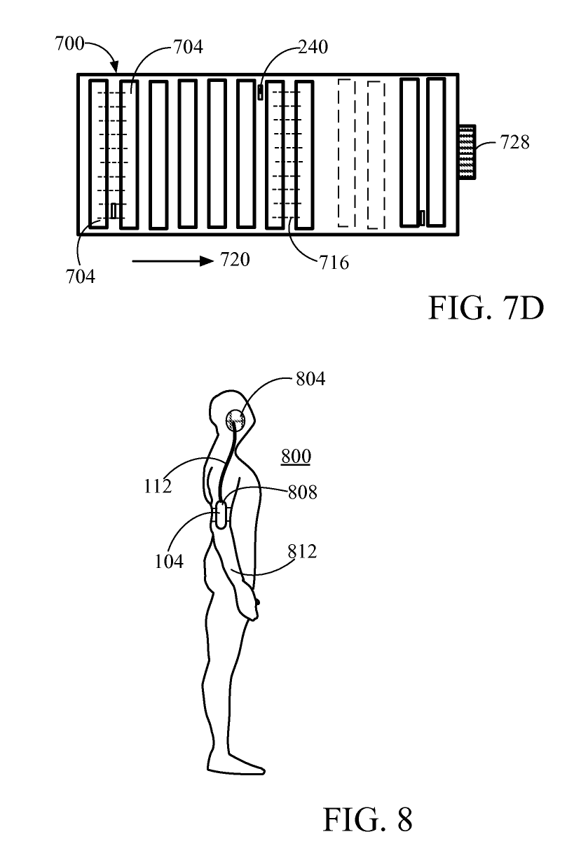

FIG. 8 is a simplified illustration of a user performing skin treatment in accordance with the current method and apparatus. User 800 applies patch 804 which may be any one of the described above patches and places case 104 that includes RF voltage generator 124 (FIG. 1) into a pouch 808 carried, for example, on the user's 800 arm 812. The user connects between patch 804 and case 104 by cable 112 and by pushing button 124 (FIG. 1) switches ON RF voltage generator 120. The user could set one of the predetermined skin treatment protocols. RF voltage generator provides test RF voltage and determines the quality of the contact between the patch electrodes and the treated segment of skin. Measurement of the impedance between the electrodes and the skin may serve as the quality of the contact between the patch electrodes and the treated segment of skin indicator. Upon determination of the electrodes status RF voltage generator may begin delivery of the treatment RF voltage to the electrodes of the patch. The treatment continues for the time set by the skin treatment protocol and the user is free address and work on other issues and tasks. If the user desires he can track the treatment process by operation status indicator 128 (FIG. 1) such as an LED that may lit with more than one color.

The employment of an applicator that includes disposable parts for electromagnetic radiation skin treatment simplifies and facilitates aesthetic treatments in a home environment at a time most convenient for the user to perform a treatment session. Use of RF energy for performing skin treatment according to a predetermined treatment protocol is safe and enables a lay user to use it in a residential environment and provides the user with freedom to perform concurrently to the treatment other tasks.

A number of embodiments have been described. Nevertheless, it will be understood that various modifications may be made without departing from the spirit and scope of the method and patch structure. Accordingly, other embodiments are within the scope of the following claims:

What is claimed is:

1. A patch for personal cosmetic skin treatment, said patch comprising:
a substrate with a plurality of electrodes operative to couple RF voltage to skin when the patch is applied to a treated skin segment; and
wherein the electrodes are coated by an electrically conductive adhesive, said adhesive enabling firm electrical and mechanical coupling of the patch and the electrodes to the treated skin segment.

2. The patch according to claim 1 wherein the substrate is one of a group of substrates consisting of polyimide film, paper, and plastic material, with a thickness of 0.5 mil to 60 mil (12.5 micron to 1500 micron).

3. The patch according to claim 1 wherein the electrodes are in electrical communication with an RF voltage supply.

4. The patch according to claim 1 wherein free of electrodes areas of the substrate are covered by an electrically isolative adhesive.

5. The patch according to claim 1 wherein the electrodes are arranged in a pattern enabling operation of the patch in a mono-polar or bi-polar operation mode; and
wherein the electrodes arranged in a pattern enabling operation of the patch in a mono-polar operation mode consist of a plurality of active and common electrodes and wherein the active electrodes surround the common electrodes.

6. The patch according to claim 1 wherein the electrodes are arranged in a pattern enabling operation of the patch in a linear sweeping operation mode.

7. The patch according to claim 1 wherein the plurality of the electrodes enable fractal RF skin treatment.

8. The patch according to claim 1 further comprising light sources delivering light of suitable wavelength to the treated skin segment.

9. The patch according to claim 1 further comprising one or more temperature sensors operative to switch RF voltage supply if skin or electrode temperature exceeds a required limit.

10. A sheet of disposable patches for personal cosmetic skin treatment, said sheet comprising:
a substrate with a plurality of electrodes located on different patch shapes with each patch surrounded by cut-out lines; and
a layer of electrically conductive adhesive covering the electrodes located on the patch shapes.

11. The sheet of disposable patches according to claim 10 further comprising a release layer covering said sheet.

12. The sheet of disposable patches according to claim 10 further comprising cut-outs enabling easy patch separation said cut-outs made in the substrate and in release layer.

13. The sheet of disposable patches according to claim 10 wherein said sheet includes patches with electrodes for conventional and fractal skin treatment.

14. The sheet of disposable patches according to claim 10 wherein the electrodes are deposited on a substrate which is one of a group of substrates consisting of polyimide film, paper, and plastic material, with a thickness of 0.5 mil to 60 mil (12.5 micron to 1500 micron).

15. A disposable patch for personal fractal cosmetic skin treatment, said patch comprising:
a substrate with at least one electrode covered by an electrically conductive adhesive, said electrode operative to couple RF voltage to skin when it is applied to the skin;
an RF voltage generator located on said substrate and operative to provide RF voltage to the at least one electrode; and
wherein the RF voltage generator becomes operative when the at least one electrode forms firm contact with the skin.

16. The patch according to claim 15 wherein said electrode coated by an electrically conductive adhesive enables firm mechanical coupling of the electrode to the skin.

17. The patch according to claim 15 further comprising a skin treatment protocol selection device operative to set a skin treatment protocol and wherein the skin treatment protocol selection device operation is initiated by configuring the skin treatment protocol selection device.

18. The patch according to claim 15 wherein the substrate is one of a group of substrates consisting of polyimide film, paper, and plastic material, with a thickness of 0.5 mil to 60 mil (12.5 micron to 1500 micron).

19. A wearable apparatus for personal cosmetic skin treatment, said apparatus comprising:
a patch according to claim 1;
a wearable RF voltage generator being in electrical communication with the electrodes of the patch and operative to supply RF voltage to the electrodes of the patch; and wherein the RF voltage is supplied in accordance with a predetermined treatment protocol.

20. The apparatus according to claim 19 wherein the predetermined treatment protocol was established in laboratory conditions.

21. The apparatus according to claim 20 wherein the predetermined treatment protocol includes supply of RF voltage in pulse or continuous mode.

22. The apparatus according to claim 21 further comprising an RF voltage generator providing at least a test RF voltage and a skin treatment RF voltage.

23. The apparatus according to claim 22 wherein the skin treatment RF voltage is supplied to the electrodes according to the predetermined treatment protocol selected to cause slow heating of skin layer volumes to a temperature not exceeding 62 degrees Celsius.
